(19) United States
(12) Patent Application Publication
(10) Pub. No.: US 2023/0227766 A1
(43) Pub. Date: Jul. 20, 2023

(54) A CULTURE PLATFORM FOR CULTIVATING TISSUE AND METHOD FOR OBSERVING TISSUE CULTIVATED THEREIN

(71) Applicant: Westfaelische Wilhelms-Universitaet Muenster, Muenster (DE)

(72) Inventors: Arne Hofemeier, Muenster (DE); Timo Betz, Muenster (DE)

(21) Appl. No.: 17/925,247

(22) PCT Filed: May 17, 2021
(86) PCT No.: PCT/EP2021/062976

§ 371 Date: Nov. 14, 2022

(30) Foreign Application Priority Data
May 15, 2020 (LU) LU101799

(51) Int. Cl. C12M 1/34; C12M 3/00; C12M 1/00

(52) U.S. Cl. CPC C12M 41/36; C12M 21/08; C12M 23/22

(57) ABSTRACT

In various embodiments a culture platform for cultivating tissue is provided, comprising: a first component (2) comprising at least one culture chamber (21) formed therein, the culture chamber (21) comprising a bottom (23), a sidewall and an open top (22); and a second component (2) comprising a base (31) and at least one pair of posts (32) extending from the base (31); wherein the first component (2) and the second component (3) are sized and configured to be mated with one another such that when the second component (3) is placed on the first component (2), the at least one pair of posts (32) is inserted into the at least one culture chamber (21). Furthermore, a method for observing tissue cultivated therein is provided.

The present invention relates to a culture platform for cultivating tissue, in particular to an in vitro muscle tissue culture platform which may be used for high resolution microscopy and functional analysis. The present invention also relates to a corresponding method for observing tissue cultivated in the culture platform.

Skeletal muscle tissue supports essential functions, e.g. breathing, swallowing and limb movement. Strategies to improve muscle function were historically tested in non-human animal models, but in recent years, there is a shift to enlisting microphysiological tissue systems to assess therapeutic efficacy in the context of a human cell assay. To date, all available 3D culture systems to produce skeletal muscle microtissues for testing putative therapies are based on polydimethylsiloxane (PDMS) molds bearing the consequence of immense chemical absorbance of any kind of proteins, thus altering the direct environment of the tissue under study.

One common characteristic of all 3D muscle microtissue culture platforms is the integration of design features that enables non-invasive measurements of tissue force generation by quantifying deflection of those features that can be captured in short videos following tissue stimulation. A prominent example of such features which are used to characterize the tissue under study includes flexible posts or pillars which are arranged at a distance from one another. When muscle tissue fibers are seeded in a chamber around two posts together with an extracellular matrix (ECM), usually 3D skeletal muscle tissues in vitro self organize around those posts. As already mentioned, the chambers which are used in such experiments are based on polydimethylsiloxane (PDMS) and plastic molds that can be tuned in their elastic properties and are soft enough to allow measurement of muscle contraction forces by recording the deflection of the posts. Such non-invasive measurements of force generation in reconstituted muscles are based on the elastic properties of PDMS.

A downside of PDMS is that poor optical properties and the required relatively large material thickness prevent the use of high numerical aperture objectives which would be required to enable high resolution microscopy and thus make modern 3D microscopy methods like confocal and spinning disk microscopy on living tissue impossible. Furthermore, for the study of a tissue with high resolution the tissue needs to be fixed, such that research on questions related to dynamical processes is also impossible with the conventional setups. This limits the application of known experimental methods of the aforementioned kind in an effort to investigate current key research questions such as cell-cell interaction or myoblast fusion. Moreover, PDMS has an immense capacity to absorb chemicals and proteins and is therefore unsuitable for serum-free medium applications and precise dose-dependent drug evaluation.

In light of the above described shortcomings, the goal of the present invention is to provide an improved culture platform for cultivating tissue, in particular muscle tissue, which may be used for high resolution imaging, precise drug screenings, diagnosis and serum-free media cultivation.

According to the present invention, in contrast to currently known experimental setups for production of muscle microtissues, skeletal muscle tissues are cultured in vitro at an inverted geometry in the sense that the tissue under study is located closely to the bottom of the culture chamber which preferably comprises a thin glass bottom, e.g. a microscopy grade glass slide. A high resolution in microscopy mandates the use of lenses that have a high numerical aperture, and these lenses in turn have short working distances. This is the main motivation for the use of (microscopy) glass and the cultivation of the tissue at the bottom of the culture chamber, i.e. in direct proximity to the transparent bottom thereof. This situation is only given when the new methodology described herein is followed, namely by cultivating the tissue under study in close proximity to the ends of posts which are arranged “upside down” as compared to the setups known from the prior art. This geometrically inverted approach allows real-time high resolution imaging while still relying on self-organization of the microtissue around the tips of the thin posts which are preferably made of polymethylmethacrylate (PMMA) and which extend downwards into the culture chamber from the top of the culture chamber. The posts allow for an accurate quantification of global forces via detection of post deflection. The culture platform of the present invention is constructed such that it may be advantageously used in combination with high numerical aperture objectives required for high resolution microscopy, thus enabling use of modern 3D microscopy methods like confocal and spinning disk microscopy on living tissue. The observation takes place through the bottom of the culture wells, while access to the culture chamber, e.g. in order to provide chemical and/or electrical stimuli to the cells, is granted through the top thereof. In that manner, the culture platform of the present invention is configured such that its bottom is optimized for and is preferably solely used for high resolution imaging while the manipulation of the microtissue provided in the culture chamber is performed through holes in the top side of the culture chamber. In the context of the present invention, a microtissue of a certain cell kind refers to a preferably scaffold-free, three-dimensional aggregate of tissue with a dimension on the microscopic scale, e.g. on the order of a few millimeters. A scaffold-free aggregate of tissue refers to a structural formation of tissue which has formed by self-organization and not by occupation of or infiltration into a structured scaffold (e.g. a 3D printed scaffold). It is noted that an ECM matrix, i.e. a three-dimensional network of extracellular macromolecules (e.g. collagen, enzymes, and glycoproteins), can be provided in the culture chamber in order to provide an environment which is conducive to the proliferation and self-organization of the cells. However, such an ECM matrix is not considered as a scaffold in the context of this description, since it provides a biochemical support for the cells rather than an “artificial” premanufactured structured scaffold which determines the final shape of the microtissue.

In various embodiments, a culture platform for cultivating tissue is provided. The culture platform, which will be also referred to as “device” in the following, comprises a first component comprising at least one culture chamber formed therein, the culture chamber comprising a bottom, a sidewall and an open top. The culture platform further comprises a second component comprising a base and at least one pair of posts extending from the base. The first component and the second component are sized and configured to be mated with one another such that when the second component is placed on the first component, the at least one pair of posts is inserted into the at least one culture chamber.

The device of the present invention may be seen to correspond to an in vitro 3D muscle tissue culture mold comprising substantially two parts which fit into one another. The first component may correspond to the bottom part of the device. In the case of multiple culture chambers being provided in the first component, the culture chambers may be provided in a regular pattern, e.g. a grid pattern, for example in a two by five or in a four by four pattern. Each culture chamber may have a certain volume defined by its bottom and its sidewall. The at least one culture chamber provides a culture well in which, for example, muscle precursor cells within an extracellular matrix may be seeded.

The second component which may be seen to correspond to the top part substantially comprises a support or a base from which long substantially vertical posts extend in a downward direction when the top part is placed on the bottom part. Overall, the number of posts is at least equal to or greater than twice the number of culture chambers such that at least a pair of posts is allocated to a given culture chamber. The second component may function as a lid. When placed on the first component, it covers the top side of the first component and the at least one culture chamber formed in the first component. The posts may be manufactured from the same or from a different material than the second component. The posts may be manufactured separately from the base of the second component and subsequently attached thereto or the whole second component (.e.g. by adhesives or by other means) may be formed integrally, i.e. manufactured as one integral piece. The posts may have a cylindrical shape with any suitable cross-sectional shape, e.g. elliptic, round, polygonal or contoured in an undulating manner. Furthermore, the tips of the posts may have clear-cut edges which allow precise determination of their deflection by a high resolution microscope. A clear cut edge may refer to an edge which is formed by a substantially 90° angle between the top surface of the post and the side surface of the post.

According to further embodiments of the culture platform, the bottom of the at least one culture chamber may comprise glass, preferably a microscopy coverslip. The glass bottom may be provided in the form of a sheet of glass attached to the bottom of the first component, thus forming the bottom of the at least one culture chamber. The use of glass as the bottom of the at least one culture chamber allows for the observation of the microtissue formed in the culture chamber by means of inverted high resolution microscopy. For example, an oil or water immersion objective may be used for that purpose in inverted orientation, i.e. with its objective arranged under the glass bottom of the first component.

According to further embodiments of the culture platform, the cross section of at least a portion of the culture chamber may have an elliptic shape. For example, a lower fraction of the culture chamber may have an elliptic shape whereas the remaining upper fraction of the culture chamber may have a differently shaped cross section, e.g. a round cross section. Here, the cross section of the culture chamber as perceived in a top view is meant, i.e. when looking into the at least one culture chamber through its top open end. Alternatively, any other cross sectional shape deemed suitable for the intended use may be chosen, such as a round shape. The elliptic shape of the at least one culture chamber may be advantageous in that, when the posts are arranged such that they are aligned along the longer of the two principal axes of the elliptic bottom shape of the culture well, more initial cell tissue material (e.g. individual muscle tissue strands) is provided along this direction than along the transverse direction. Thus, a bias may be provided to assist the self organization process by which the final annular muscle microtissue is formed which “wraps around” the ends of the posts.

According to further embodiments of the culture platform, the first component may comprise a block, preferably a cuboid, in which the at least one culture chamber is provided. Expressed differently, the first component of the device may be provided in the form of a block of a material in which at least one culture chamber (preferably a number of culture chambers) is formed. The first component may be manufactured by various processes, such as injection molding or by a cutting process such as drilling.

According to further embodiments of the culture platform, the material from which the first component is manufactured may comprise polymethylmethacrylate (PMMA), also known as acrylic glass. Preferably, the first component of the device may be manufactured from glass and acrylic glass, exclusively. This choice of materials enables both high resolution microscopy through the glass bottom and global force measurements without any detrimental protein adsorption. The use of acrylic glass and glass offers those advantages in particular over corresponding systems which are based on polydimethylsiloxane (PDMS) and suffer from both low optical properties and severe adsorption of serum derived proteins like growth factors and signaling molecules.

According to further embodiments of the culture platform the length of each of the at least one pair of posts may be such that when the second component is placed on the first component, each one of the at least one pair of posts extends substantially down to the bottom of the culture chamber. Such a configuration may provide for an optimized formation of the 3D muscle microtissue around the tips of the posts.

According to further embodiments of the culture platform, each of the posts may have a diameter of 1 mm or less. This dimension of the posts may be chosen in order for the posts to have a suitable rigidity such that deformation of the posts may be observed by means of microscopy when the muscle microtissue exerts force on the posts. In general, the rigidity of a post may be adjusted by its length, measured from the point where it emerges from or is attached to the base of the second component, and by its thickness. Cultured skeletal muscle tissues show fundamental features of functional skeletal muscle tissues such as striated multinucleated myotube pattern and the ability to react on various stimuli, e.g. optogenetically or electrically induced twitches/contractions or chemically induced acetylcholine tetanus contractions. The device of the present invention provides an easy and reliable readout for tissue strength during contraction by determining the deflection of the posts extending from the base of the second component of the device.

According to further embodiments of the culture platform, the first component may comprise at least one first alignment element and the second component may comprise at least one corresponding second alignment element, wherein the first and second alignment elements are configured to interact with one another such that they define the position in which the second component comes to rest on the first component when the second component is placed thereon. An exemplary alignment system including the first and second alignment elements may include at least one, preferably two rods, arranged on the same side of the second component as the posts and extending from the second component in the same direction as the posts. At the same time, the first component may correspondingly comprise at least one, preferably two openings which are arranged on the same side of the first component as the open tops of the at least one culture chamber. The at least one opening has a size and shape that is matched to the dimension of the at least one rod such that the at least one rod fits tightly into the corresponding opening. When the at least one rod is placed into the corresponding opening in the first component it slides into the opening when the second component is mated with the first component. In that manner, this mechanism functions as an alignment or guiding rail and assists the lowering of the second component onto the first component and defines the final position of the first component and the second component relative to one another. A precise alignment of the first component relative to the second component enables a precise alignment of the at least one pair of posts within a corresponding culture well and provides for a good replicability of the alignment of the rods within the culture well. With a precise manufacturing of the alignment elements and their location on the first and second component, respectively, a submicron precision of the alignment of the first component and the second component relative to one another may be achieved. It is noted that the alignment system described in this paragraph is only one exemplary alignment system which allows for a precise alignment of the two components of the system relative to one another. Other suitable systems providing that same functionality may be used as well.

According to further embodiments of the culture platform, the base of the second component may comprise at least one through opening which is arranged in a region above the at least one culture chamber, for example above the bottom of the at least one culture chamber, when the second component is placed on the first component. In other words, the at least one through opening may be provided in a region of the base of the second component which is arranged above the culture well when the second component is placed on the first component. The at least one through opening may be used for gas exchange and/or to change the cell culture medium and/or add chemicals/drugs inside the culture well and/or or to introduce electrodes into the culture chamber.

According to further embodiments of the culture platform, the first component may comprise multiple culture chambers provided therein and the second component may comprise, correspondingly, multiple pairs of posts such that when the second component is placed on the first component, each pair of the posts is inserted into the corresponding culture chamber.

The device of the present invention may be used for the analysis of 3D microtissue, in particular 3D skeletal muscle microtissue in vitro which self organizes around posts. In contrast to devices which allow for analysis of muscle microtissue by measurement of muscle contraction forces by recording the deflection of the posts under force exerted by the microtissue, those devices have the disadvantage that the annular microtissue structure which has formed around the posts extending upwards from the bottom tends to “climb” up the posts and ultimately disengages from the posts. In such a case, the experimental setup is not usable any more. In this context it is important to keep in mind that in the prior art the muscle microtissue structure forms around the bases of the posts. The climbing motion of the annular muscle microtissue structure is made possible by the fact that the contractive force exerted upon the two posts deflects the posts towards one another and consequently the distance between the posts grows smaller with growing distance from the base of the posts. Thus, in an exaggerated image, the posts provide an upward slide for the annular muscle microtissue structure and since the posts stand erect freely from their base, the counterforce acting against their deflection provided by stiffness of the posts grows smaller with growing distance from the base of the posts. In this exaggerated image it is easy to realize that a moment of force directed upwards is generated which pushes the annular muscle microtissue structure upwards thus enabling the microtissue to climb up the converging rungless “ladder” provided by the deflected posts. Most importantly, there is no mechanism which provides a force counteracting the deflection of the posts at the tips of the posts.

The inventors of the present culture platform have realized that this problem may be at least greatly mitigated if not avoided at all by inverting the geometry of the system. By inserting the posts into a culture chamber in which the muscle tissue is seeded together with the ECM from the top of the culture chamber has an unforeseen impact on the whole experimental setting. Once the annular muscle microtissue structure has been formed and exerts force on the posts, the posts are deflected towards one another. However, since the posts extend downwards from the base of the second component arranged at the top of the culture well, the force counteracting deflection of the posts grows larger with growing distance from the bottom of the culture well—or with decreasing distance from the base of the second component from which the posts extend. In addition, in contrast to prior art, a downward directed moment of force acts on the annular muscle microtissue structure when the annular muscle microtissue structure tries to climb up the divergent (rungless) “ladder”. This moment of force not only prevents the microtissue structure from moving upwards, i.e. towards the top of the culture chamber and at the same time away from the bottom of the culture well, but it also exerts a downward force which, in fact, keeps the microtissue structure close to the bottom of the culture chamber which is additionally advantageous from the point of view of the observation of the microtissue via high resolution microscopy. Therefore, the culture platform of the present invention provides an inherent “clamping mechanism” in the sense that, by design, it prevents a migration and disengagement of the muscle microtissue structure from the posts and keeps it close to the bottom of the culture chamber. Overall, the culturing system of the present invention allows for a variety of subcellular studies for the first time, e.g. cellular interaction assays, which were not possible in previous devices due to the above-mentioned shortcomings relating to poor optical properties and the inability to use high numerical aperture objectives required for high resolution microscopy.

In further embodiments a method for observing tissue provided in the at least one culture chamber of the culture platform according to the invention is provided, wherein the second component is arranged or placed on the first component with the at least one pair of posts being inserted in the at least one culture chamber. The method comprises observing the tissue provided in the at least one culture chamber of the culture platform through the bottom of the culture chamber.

According to further embodiments, the method may further include placing the culture chamber in an inverted microscope or positioning a microscope such that the inside of the culture chamber may be observed by means thereof through the bottom of the culture chamber. In particular, high numerical aperture objectives for high resolution microscopy may be employed in order to observe the microtissue inside the culture chamber, for example by means of confocal and spinning disk microscopy. In combination with the use of (microscopy) glass as the material from which the bottom of the culture chamber is formed enables then use of high numerical aperture objectives, for example ≥40× water/oil lenses, which facilitate high resolution microscopy of subcellular structures (e.g. cell nucleus ≤10 μm, sarcomere structures ≤1 μm).

According to further embodiments, the method may further include, as a preceding preparatory step, the preparation of a 3D microtissue in the cell chamber by seeding the cells or strands of cells of the microtissue in the culture chamber together with an extracellular matrix and allowing the formed tissue to in vitro self organize around the posts provided in the culture chamber.

According to further embodiments of the method, the tissue provided in the at least one culture chamber may comprise muscle tissue.

According to further embodiments of the method, observing the tissue provided in the at least one culture chamber includes registering (measuring) differences in distance between the pair of posts located in the culture chamber. The evaluation of the change of the distance between the ends of a pair of posts allows for a measurement of muscle contraction forces by recording the deflection of the posts. Such non-invasive measurements of force generation in reconstituted muscles are based on the elastic properties of the material, preferably PMMA, from which the posts are manufactured.

Summarizing the main aspect of the present invention, the cultivation of the tissue under study at the bottom of the at least one culture chamber combined with the use of posts in an “overhead” or inverted arrangement enables to maintain the microtissue under study in close proximity to (microscopy) glass bottom of the culture chamber. The cultivation of tissue on (microscopy) glass enables the use of high-resolution lenses which facilitate investigation of dynamic subcellular processes during tissue development. The use of posts in an overhead arrangement has no disadvantages whatsoever as compared to approaches of the prior art where the posts are attached to a base on which the microtissue under study is formed, since apart from cultivation, all force measurements can be carried out in the same way as in those conventional setups. The force measurements can even be carried out more precisely, since the deformable posts are manufactured with clean-cut edges and can be imaged with a much higher resolution due to the possibility of using high numerical aperture objectives. Consequently, even the smallest forces can be measured.

In the following, the device of the present invention will be described in more detail with reference to the appended figures.

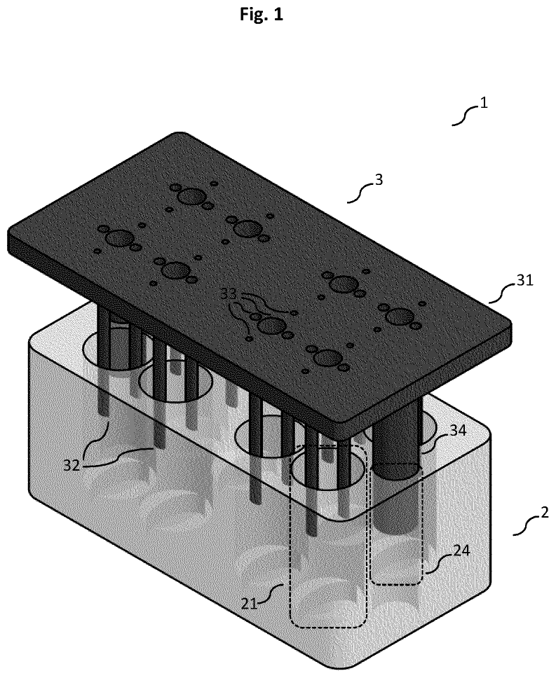

FIG. 1 shows a perspective view of the culture platform according to various embodiments of the invention.

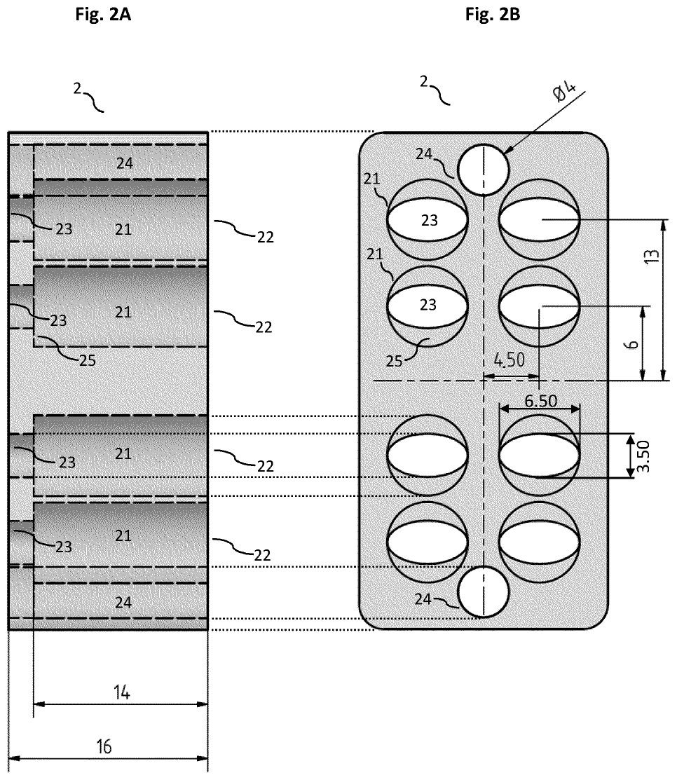

FIG. 2A shows a cross-sectional side view of the first component of the culture platform according to various embodiments of the invention.

FIG. 2B shows a top view of the first component of the culture platform according to various embodiments of the invention.

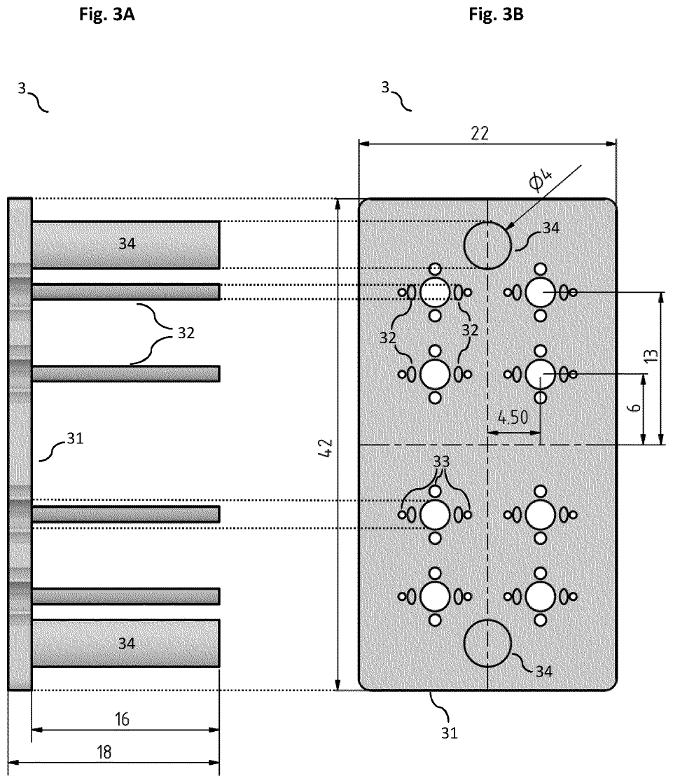

FIG. 3A shows a cross-sectional side view of the second component of the culture platform according to various embodiments of the invention.

FIG. 3B shows a top view of the second component of the culture platform according to various embodiments of the invention.

In FIG. 1, a perspective view of the culture platform 1 according to various embodiments is shown. In FIGS. 2A-3B, detailed cross-sectional side views and top views of the first component 2 and the second component 3 are shown. It is noted that the dimensions of various elements of the first component 2 and the second component 3 as given in FIG. 2B and FIG. 3B, respectively, are exemplary values which are provided to obtain a better overall image of the culture platform 1 according to various embodiments. The exemplary values are by no means understood as exact parameters which are necessary to execute the invention.

The culture platform 1 for cultivating tissue shown in the figures comprises the first component 2 with a total of eight culture chambers 21 formed therein. The culture chambers 21 are arranged in two rows, each row having four culture chambers 21. Each culture chamber 21 comprises a bottom 23, a sidewall and an open top 22. In the exemplary embodiment of the culture platform 1 shown in the figures the bottom 23 of each culture chamber 21 has an elliptic shape. Furthermore, each culture chamber 21 has a staggered bottom in the sense that the shape of the bottom 23 of the culture chamber 21 defines a first volume which opens into second volume which is larger than the first volume to maximize the amount of medium that can be applied to the culture well 21. The size and height of the first volume may vary to change the final volume of the microtissue depending on the respective requirements. The second volume has a cylindrical shape with a round ground surface which corresponds to the opening 22 of the culture chamber 21. At the transition between the smaller first volume with the elliptic ground surface and the larger second volume with the round ground surface two steps 25 are formed. The bottoms 23 of the culture wells 21 comprise glass which may be provided in the form of at least one piece of glass, or example a microscopy coverslip, and be attached to the bottom of the first component 2.

The culture platform 1 further comprises the second component 3 comprising a base 31 and a number of pairs of posts 32 extending from the base 31. The first component 2 and the second component 3 are sized and configured to be mated with one another such that when the second component 3 is placed on the first component 2, each pair of posts 32 is inserted into a corresponding culture chamber 21. Each pair of posts 32 is arranged such that when the second component 3, also functioning as a lid, is placed on the first component 2, the tips of a pair of posts 32 are arranged within the surface corresponding to the bottom 23 of the corresponding culture chamber 21.

The culture platform 1 further comprises an alignment and/or positioning mechanism which relies on the interaction between first alignment elements provided in the first component 2 and second alignment elements provided in the second component 3. In this example, each of the first alignment elements corresponds to an alignment opening 24. Each of the alignment openings 24 is sized and arranged to receive an alignment rod 34. Placing the second component 3 on the first component 2 results in the alignment rods 34 sliding into the alignment openings 24. In that manner, the interaction between the alignment openings 24 and the alignment rods 34 guarantees that each time the position in which the second component 3 comes to rest on the first component 2 is the same. It is noted that more that the principle of the alignment mechanism may be also realized by using more than two rod-opening pairs. In further embodiments it is also possible to use only one rod-opening pair. In such a case the cross-section of the alignment opening 24 and, correspondingly, the alignment rod 34 would have a shape which prevents rotation of the alignment rod 34 within the alignment opening 24, such as a polygonal or other shape. Implementing two or more rod-opening pairs obviously allows for round alignment elements since the rotation of the first component 2 relative to the second component 3 is not possible.

In order to be able to access the inside of the culture chambers 21 once the second component 3 has been placed on the first component 2, the second component 3 comprises openings 33 of various shapes and sizes which are arranged in a region above the culture chamber 21 when the second component 3 is placed on the first component 2.

1. A culture platform (1) for cultivating tissue, comprising:
a first component (2) comprising at least one culture chamber (21) formed therein, the culture chamber (21) comprising a bottom (23), a sidewall and an open top (22); and
a second component (2) comprising a base (31) and at least one pair of posts (32) extending from the base (31);
wherein the first component (2) and the second component (3) are sized and configured to be mated with one another such that when the second component (3) is placed on the first component (2), the at least one pair of posts (32) is inserted into the at least one culture chamber (21); and
wherein the bottom (23) of the at least one culture chamber (21) comprises glass.

2. Culture platform (1) of claim 1, wherein the bottom (23) of the at least one culture chamber (21) comprises a microscopy coverslip.

3. Culture platform (1) of claim 1 or 2, wherein the cross section of at least a portion of the culture chamber (21) has an elliptic shape.

4. Culture platform (1) of any one of claims 1 to 3, wherein the first component (2) comprises a block, preferably a cuboid, in which the at least one culture chamber (21) is provided.

5. Culture platform (1) of any one of claims 1 to 4, wherein the material from which first component (2) is manufactured comprises PMMA.

6. Culture platform (1) of any one of claims 1 to 5, wherein the length of each of the at least one pair of posts (32) is such that when the second component (3) is placed on the first component (2), each one of the at least one pair of posts (32) extends substantially down to the bottom (23) of the culture chamber (21).

7. Culture platform (1) of any one of claims 1 to 6, wherein each of the posts (32) has a diameter of 1 mm or less.

8. Culture platform (1) of any one of claims 1 to 7, wherein the first component (2) comprises at least one first alignment element (24) and the second component (3) comprises at least one corresponding second alignment element (34), wherein the first and second alignment elements (24, 34) are configured to interact with one another such that they define the position in which the second component (3) comes to rest on the first component (2) when placed thereon.

9. Culture platform (1) of any one of claims 1 to 8, wherein the base (31) of the second component (3) comprises at least one through opening (33) which is arranged in a region above the at least one culture chamber (21) when the second component (3) is placed on the first component (2).

10. Culture platform (1) of any one of claims 1 to 9, wherein the first component (2) comprises multiple culture chambers (21) provided therein and the second component (3) comprises, correspondingly, multiple pairs of posts (32) such that when the second component (3) is placed on the first component (2), each pair of the posts (32) is inserted into the corresponding culture chamber (21).

11. Method for observing tissue provided in the at least one culture chamber (21) of the culture platform (1) according to any one of claims 1 to 10, wherein the second component (2) is arranged on the first component (3) with the at least one pair of posts (32) being inserted in the at least one culture chamber (21), the method comprising:
observing the tissue provided in the at least one culture chamber (21) of the culture platform (1) through the bottom (23) of the culture chamber.

12. Method of claim 12, wherein the tissue provided in the at least one culture chamber (21) comprises muscle tissue.

13. Method of claim 12, wherein observing the tissue provided in the at least one culture chamber (21) includes registering differences in distance between the pair of posts (32) located in the culture chamber (21).
